(19) United States
(12) Patent Application Publication
(10) Pub. No.: US 2004/0066939 A1
(43) Pub. Date: Apr. 8, 2004

(54) Stethoscope ear piece

(76) Inventor: Wan-Chen Wong, Taipei (TW)

Correspondence Address: Wan-Chen Wong, PO Box 82-144, Taipei, TW

(21) Appl. No.: 10/636,663

(22) Filed: Aug. 8, 2003

(30) Foreign Application Priority Data
Oct. 8, 2002 (CN) 02 2 57233.3

(51) Int. Cl. A61B 7/04; H04R 25/00; A61B 7/02

(52) U.S. Cl. 381/67; 381/328; 181/135

(57) ABSTRACT

An improved structure of a stethoscope earpiece is essentially comprised of an ear nib containing an insertion part to receive a bolt and the ear nib made of resilient rubber being locked to a terminal of a branch extended from the stethoscope by means of the bolt characterized by that a ring groove filled with liquid solution is provided in the ear nib to make the earpiece more comfortable to wear for longer time.

### BACKGROUND OF THE INVENTION

(a) Technical Field of the Invention

The present invention is related to an improved structure of a stethoscope earpiece, and more particularly, to one that is provided with a hollow ring groove containing liquid solution to improve softness of the ear piece for comfort in long-term wearing.

(b) Description of the Prior Art

A stethoscope provided with a pair of earpieces is an auscultation apparatus a physician has to wear between his ears while attending a patient. As illustrated in FIGS. 1, 2 and 3 of the accompanying drawings, the earpiece is essentially comprised of an ear nib (1) and a threaded bolt (20) to be connected to a terminal of a U-shape branch of the stethoscope. A hose (14) in proper length is provided inside the ear nib and an adapter (15) is provided at the end of the hose (14). An arc ball (10) extended from the front end of the hose (14) contains a hollow space (16) provided with a thin wall (11) for the transmission of sound wave generated through the hose (14) and the U-shape branches of the stethoscope in the course of auscultation. However, the conventional ear piece is made of hard rubber, it prevents from the wall from creating a proper resilience even though a larger hollow space is formed inside the ear piece. Plugging such a hard earpiece in the ear for longer hours will cause the ear to get uncomfortable if not sour or pain. The prior art of the earpiece for the stethoscope generally available in the market has been for years sought any improvement in terms of the discomfort problem of wearing the stethoscope for longer hours.

### SUMMARY OF THE INVENTION

The primary purpose of the present invention is to provide an improved structure of a stethoscope earpiece that is resilient to make the earpiece more comfortable to wear for longer hours. To achieve the purpose, the present invention is essentially comprised of an ear nib provided with an adapter to be secured to a terminal of the stethoscope by means of a bolt; and the ear nib is made of a resilient rubber integrated with an arc groove in the thin wall of the ear-nib and a liquid solution is filled in the arc groove during the integration.

The foregoing object and summary provide only a brief introduction to the present invention. To fully appreciate these and other objects of the present invention as well as the invention itself, all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is a sectional view of the prior art.

FIG. 2 is a plane view of the prior art.

FIG. 3 is a perspective view of the prior art.

FIG. 4 is a sectional view of a preferred embodiment of the present invention.

FIG. 5 is a plane view of the preferred embodiment of the present invention.

FIG. 6 is a perspective view of the preferred embodiment of the present invention.

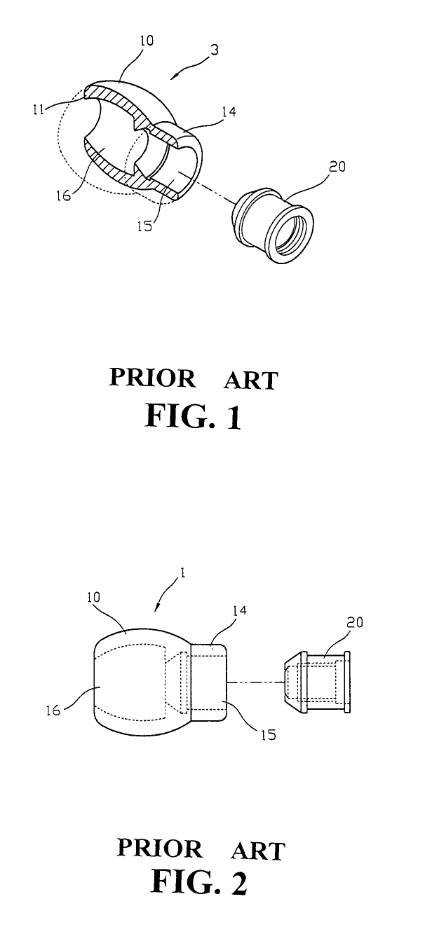

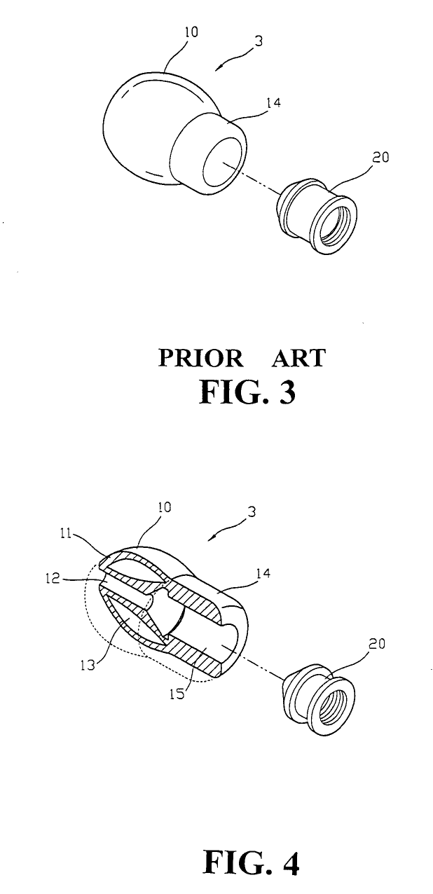

### DETAILED DESCRIPTION OF THE PREFERRED EMBODIMENT

The following descriptions are of exemplary embodiments only, and are not intended to limit the scope, applicability or configuration of the invention in any way. Rather, the following description provides a convenient illustration for implementing exemplary embodiments of the invention. Various changes to the described embodiments may be made in the function and arrangement of the elements described without departing from the scope of the invention as set forth in the appended claims.

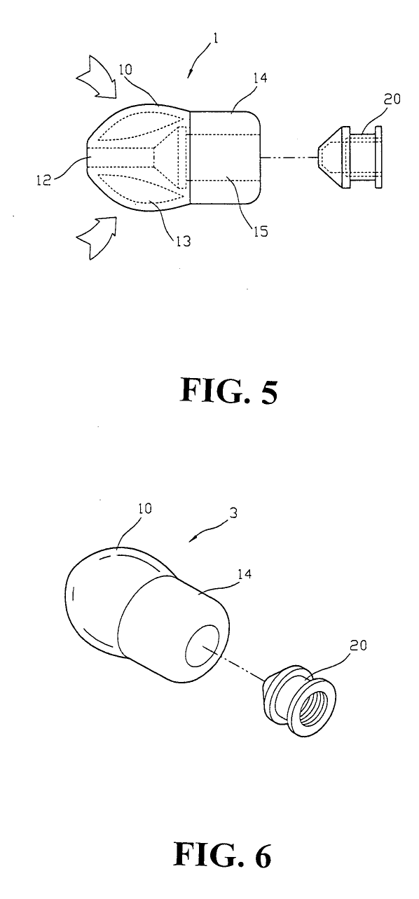

Referring to FIGS. 4, 5, and 6, a preferred embodiment of the present invention is essentially comprised of an ear nib (1) provided with an adapter (15) to receive the insertion of a bolt (20). The bolt (20) internally threaded engages a terminal of a U-shape branch extending from the stethoscope for the transmission of sound wave produced in the course of auscultation.

The ear nib (1) is made of resilient rubber and the adapter (15) to be inserted with the bolt (20) is provided at the end of a hose (14) in proper length. The interior of the adapter (15) is made in a shape to compromise that of the bolt (20). An arc ball (10) is provided at the front end of the hose (14) and a circular through hole is disposed in the arc ball (10) to connect through the adapter (15). A rubber thin wall (11) is reserved between the circular hole (12) and the arc ball (10) and a hollow ring groove (13) is integrated with ear nib (1) and filled with a liquid solution at the same time. The inherited resilience plus the ring groove (13) filled with the liquid solution offer the ear nib (1) the resilient and restoration features to compromise the arc of the human ear for the earpiece to be more comfortable to wear for longer hours.

The present invention is innovative and practical by eliminating flaws found with the prior art to provide a more comfortable wearing of the earpiece.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. An improved structure of a stethoscope earpiece, essentially comprised of an ear nib made of a resilient rubber, an adapter, and a bolt; the adapter being internally threaded and disposed in the ear nib; and the ear nib being secured to a terminal of a U-shape branch extending from the stethoscope by means of the bolt characterized by that a hollow ring groove being integrated with the ear nib; the ring groove being filled with a liquid solution to provide additional resilience for the earpiece to make it more comfortable to wear for longer hours.

2. An improved structure of a stethoscope earpiece as claimed in claim 1, wherein, an air may be filled and released in and from the hollow ring groove of the ear nib.
